(19) United States
(12) Patent Application Publication
(10) Pub. No.: US 2012/0179157 A1
(43) Pub. Date: Jul. 12, 2012

(54) SYSTEMS AND METHODS FOR SCREEN ELECTRODE SECUREMENT

(76) Inventors: Andrew Frazier, Sunnyvale, CA (US); Johnson E. Goode, Austin, TX (US); David A. Cox, Austin, TX (US); Philip M. Tetzlaff, Austin, TX (US)

(21) Appl. No.: 12/985,449

(22) Filed: Jan. 6, 2011

(51) Int. Cl. A61B 18/14; H01R 43/16

(52) U.S. Cl. 606/41; 29/874

(57) ABSTRACT

Systems and methods for securing a screen-type active electrode to the distal tip of an electrosurgical device used for selectively applying electrical energy to a target location within or on a patient's body. A securing electrode is disposed through the screen electrode and mechanically joined to an insulative support body while also creating an electrical connection and mechanical engagement with the screen electrode. The electrosurgical device and related methods are provided for resecting, cutting, partially ablating, aspirating or otherwise removing tissue from a target site, and ablating the tissue in situ. The present methods and systems are particularly useful for removing tissue within joints, e.g., synovial tissue, meniscus, articular cartilage and the like.

### FIELD OF THE INVENTION

The present invention relates generally to the field of electrosurgery, and more particularly to apparatus and methods for applying high frequency voltage to ablate tissue. More particularly, the present invention relates to apparatus and methods for securing a substantially flat screen-type active electrode to the distal tip of the shaft of an electrosurgical instrument.

### BACKGROUND OF THE INVENTION

Conventional electrosurgical methods are widely used since they generally achieve hemostasis and reduce patient bleeding associated with tissue cutting operations while improving the surgeon's visibility of the treatment area. Many of the electrosurgical devices used in electrosurgery make use of a screen-type active electrode which is typically cut, or etched, from a sheet of conductive material. These electrosurgical devices and procedures, however, suffer from a number of disadvantages. For example, screen-type active electrodes typically require some method of securement to an insulative body and furthermore to the distal tip of the device itself. Failure to adequately secure the screen electrode to the insulative body may result in improper device function and possible patient harm during the electrosurgical procedure.

Prior attempts to secure the screen active electrode to the insulative body have involved mechanical, thermal, and chemical means or various combinations thereof. Numerous mechanical forms of securement have been utilized, while adhesives have been used as a chemical form of joining, and welding the screen may provide one thermal method of joining. These mechanical joining methods may also include the use of plastic, or non-recoverable, deformations of the materials being used for securement. However, even in combination with other joining methods, the above-listed methods for fixation provide only marginally effective solutions that typically are challenged over extended periods of use.

Accordingly, devices and methods which allow for the securement of flat screen active electrodes to the insulative body of an electrosurgical instrument while maintaining electrical connections through the insulative body are desired. In particular, mechanical methods for providing reasonable and durable securement of an electrically connected screen active electrode to the insulative body at the distal tip of an electrosurgical device while providing enhanced electrosurgical operating parameters are desired.

### SUMMARY OF THE INVENTION

The present invention provides systems, apparatus and methods for mechanically securing a screen type active electrode to the insulative body at the distal tip of an electrosurgical device. In particular, methods and apparatus are provided for reliably securing the screen electrode over extended periods of use. Further, the methods and systems of the present invention are particularly useful for providing expanded and enhanced electrosurgical operating parameters.

In one aspect of the invention, the method of securement comprises inserting a securing electrode through a channel or slot in both the screen electrode and insulative body. In a configuration where the screen electrode is supported by the insulative body, the securing electrode functions to mechanically couple the screen electrode to the insulative body, and are also electrically coupled the screen electrode and a high frequency power supply via electrical connectors. Thus, the securing electrode provides a mechanical method of joining the screen electrode to the insulative body while also allowing for an electrical connection to transmit RF energy from the screen electrode to the securing electrode.

Another configuration of the electrosurgical device according to the present disclosure comprises an active screen electrode having at least two bilateral channels therethrough. At least two bilateral securing electrodes are provided and are respectively inserted through the channels of the screen electrode. Additionally, the device comprises an insulative support member having at least two bilateral channels correspondingly positioned with regard to the screen electrode channels. The bilateral securing electrodes are inserted through the support member and screen electrode channels and may be oriented symmetrically to thereby allow for creation of a zone for RF ablation between the two securing electrodes.

In certain configurations, the securing electrodes may be characterized by a saw tooth pattern on a superior surface. Additionally, the securing electrodes may be formed in the shape of a staple or bridge, thereby allowing for the creation of another zone of RF ablation in a space between the staple securing electrode and the screen electrode. The added edges formed on the securing electrode in these configurations may result in increased current density and thus promote the formation of improved zones of RF ablation.

In yet another configuration, the active electrode comprises a conductive screen having a plurality of holes and is positioned over the insulative body at the distal tip of an electrosurgical device in relation to the distal opening of an aspiration lumen. In the representative embodiment, the screen electrode is supported by the insulating support member such that the one of the plurality of holes on the screen is aligned with the aspiration lumen opening, thereby allowing for the aspiration of unwanted tissue and electrosurgery byproducts from the target site. Additionally, the screen and the distal opening of the aspiration lumen may be positioned on a lateral side of the instrument (i.e., facing 90 degrees from the instrument axis).

In open procedures, the system may further include a fluid delivery element for delivering electrically conducting fluid to the active electrode(s) and the target site. The fluid delivery element may be located on the instrument, e.g., a fluid lumen or tube, or it may be part of a separate instrument. Alternatively, an electrically conducting gel or spray, such as a saline electrolyte or other conductive gel, may be applied to the tissue. In addition, in arthroscopic procedures, the target site will typically already be immersed in a conductive irrigant, i.e., saline. In these embodiments, the apparatus may not have a fluid delivery element. In both embodiments, the electrically conducting fluid will preferably provide a current flow path between the active electrode terminal(s) and the return electrode(s). In an exemplary embodiment, a return electrode is located on the instrument and spaced a sufficient distance from the active electrode terminal(s) to substantially avoid or minimize current shorting therebetween and to isolate the return electrode from tissue at the target site.

In another aspect of the invention, a method comprises positioning one or more active electrode(s) (which may include an active screen electrode and securing electrode) at the target site within a patient's body and applying a suction force to a tissue structure to draw the tissue structure to the active electrode(s). High frequency voltage is then applied between the active electrode(s) and one or more return electrode(s) to ablate the tissue structure. Typically, the tissue structure comprises a flexible or elastic connective tissue, such as synovial tissue. This type of tissue is typically difficult to remove with conventional mechanical and electrosurgery techniques because the tissue moves away from the instrument and/or becomes clogged in the rotating cutting tip of the mechanical shaver or microdebrider. The present invention, by contrast, draws the elastic tissue towards the active electrodes, and then ablates this tissue with the mechanisms described above.

In another aspect of the invention, an electrosurgical instrument is disclosed for applying energy to a target site within or on a patient's body. The instrument has a shaft with a proximal end and a distal end and an active electrode assembly disposed at the distal end of the instrument shaft. The active electrode assembly includes a substantially flat active screen electrode and at least one securing electrode, which is in electrical contact with the screen electrode. The instrument also has at least one electrically conductive connection element, mechanically and electrically coupled to the screen electrode proximal end, and the connection element is part of the electrical conduit between the active electrode assembly and an energy source. The instrument also includes an electrically insulating support member upon which the screen electrode is mounted and the support member has at least one securing electrode slot to receive a stem of the at least one securing electrode. The support member also has at least one conductive connection element opening to receive a portion of the conductive connection element. In some aspects of the invention, the instrument may also have at least one return electrode positioned on the shaft or as part of the shaft itself, and spaced away from the active electrode assembly. The insulative support member may also be formed as a one piece component, formed from a material operable to be resistant to electrosurgical plasma degradation such as an inorganic material (i.e., ceramic).

In another aspect of the invention, an electrosurgical instrument is disclosed for applying energy to a target site within or on a patient's body, the instrument having a shaft defining a proximal end and a distal end and an insulating support member disposed at shaft distal end. The insulating support member includes a screen electrode support surface, at least one securing electrode opening on the screen electrode support surface, and at least one conductive connection element channel adjacent to the screen electrode support surface. An active electrode assembly is also disposed adjacent to the insulating support member, with a substantially flat active screen electrode disposed on the screen electrode support surface and the active assembly also includes at least one securing electrode, located adjacent the support member securing electrode opening. The active electrode assembly also includes at least one electrically conductive connection element electrically connected with the screen electrode proximal end, and a portion of the conductive connection element may be inserted into the conductive connection element channel.

In yet another aspect of the invention, a method for securing a substantially flat active screen electrode to a distal end of an electrosurgical instrument is disclosed, the method including the steps of electrically connecting an electrically conductive connection element to the proximal end of the screen electrode, followed by inserting the connection element into a support member connection element channel. The screen electrode is then disposed upon the support member and at least one stem portion of a securing electrode is extended through the screen electrode and into a slot in the support member. This stem portion is then fixedly engaged within the slot in the electrically insulating support member, so that the securing electrode mechanically secures the screen electrode to the support member. The securing electrode is also electrically connected to the screen electrode. The connection element is then electrically coupled to an electrical connector that is electrically connected with a high frequency power supply. In some aspects of this disclosure, the step of inserting the connection element may involve bending the connection element so as to insert the connection element into the channel.

### BRIEF DESCRIPTION OF THE DRAWINGS

FIG. 1 is a perspective view of an electrosurgical system incorporating a power supply and an electrosurgical probe;

FIG. 2 is a perspective view of another electrosurgical system incorporating a power supply, an electrosurgical probe and a supply of electrically conductive fluid for delivering the fluid to the target site;

FIG. 3 is a side view of an electrosurgical probe for ablating and removing tissue;

FIG. 4 is a cross-sectional view of the electrosurgical probe of FIG. 3;

FIG. 5 is a detailed view illustrating ablation of tissue;

FIG. 6 is an exploded view of the distal end portion of the probe of FIG. 3 according to at least some embodiments;

FIG. 7 is a perspective view of the distal end portion of a probe according to at least certain embodiments described in the present disclosure; and

FIG. 8 is a perspective view of the securing electrodes and screen electrode.

### DETAILED DESCRIPTION OF THE INVENTION

The present invention provides systems and methods for selectively applying electrical energy to a target location within or on a patient's body. The present invention is particularly useful in procedures where the tissue site is flooded or submerged with an electrically conducting fluid, such as arthroscopic surgery of the knee, shoulder, ankle, hip, elbow, hand or foot. In other procedures, the present invention may be useful for collagen shrinkage, ablation and/or hemostasis in procedures for treating target tissue alone or in combination with the volumetric removal of tissue. More specifically, the embodiments described herein provide for electrosurgical devices characterized by a substantially flat screen active electrode disposed at the distal tip of the device. Additionally, the present embodiments include apparatus and methods for the mechanical securement of the screen electrode to the insulative body located at the distal tip of the device. Such methods of mechanical securement of the screen electrode may extend the operating period of the electrosurgical device by providing a more secure method of attachment.

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except in so far as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The electrosurgical device of the present embodiments may have a variety of configurations as described above. However, at least one variation of the embodiments described herein employs a treatment device using Coblation® technology.

As stated above, the assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the tip of the active electrode(s) and the target tissue. The electrically conductive fluid may be a liquid or gas, such as isotonic saline, blood, extracellular or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer. Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of these phenomena can be found in commonly assigned U.S. Pat. No. 5,697,882, the complete disclosure of which is incorporated herein by reference.

In some applications of the Coblation® technology, high frequency (RF) electrical energy is applied in an electrically conducting media environment to shrink or remove (i.e., resect, cut, or ablate) a tissue structure and to seal transected vessels within the region of the target tissue. Coblation® technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons. Since different tissue structures have different molecular bonds, the Coblation® device may be configured to produce energy sufficient to break the molecular bonds of certain tissue but insufficient to break the molecular bonds of other tissue. For example, fatty tissue (e.g., adipose) has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of these phenomena can be found in commonly assigned U.S. Pat. Nos. 6,355,032; 6,149,120 and 6,296,136, the complete disclosures of which are incorporated herein by reference.

The active electrode(s) of a Coblation® device may be supported within or by an inorganic insulating support member positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

Further discussion of applications and devices using Coblation® technology may be found in commonly assigned U.S. Pat. Nos. 6,296,638; 7,241,293 and 7,429,260, each of which are incorporated herein by reference.

In one example of a Coblation® device for use with the presently-described embodiments, the return electrode of the device is typically spaced proximally from the active electrode(s) a suitable distance to avoid electrical shorting between the active and return electrodes in the presence of electrically conductive fluid. In many cases, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

A Coblation® treatment device for use according to the present descriptions may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In certain configurations, each individual active electrode in the electrode array may be electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form selected active electrodes (e.g., titanium or a resistive coating on the surface of metal, such as platinum).

The Coblation® device is not limited to electrically isolated active electrodes, or even to a plurality of active electrodes. For example, in certain embodiments the array of active electrodes may be connected to a single lead that extends through the catheter shaft to a power source of high frequency current.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck.

The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation).

Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source of the present invention delivers a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly voltages around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. In a presently preferred embodiment of the present invention, current limiting inductors are placed in series with each independent active electrode, where the inductance of the inductor is in the range of 10 uH to 50,000 uH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current-limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood).

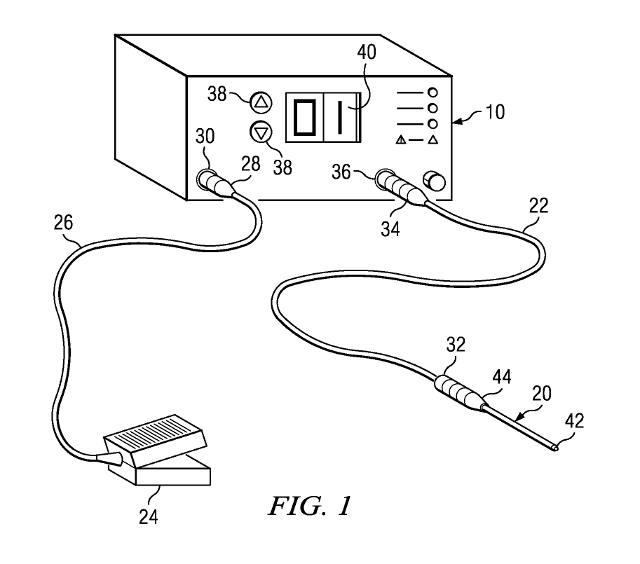

Referring now to FIG. 1, an exemplary electrosurgical system for resection, ablation, coagulation and/or contraction of tissue will now be described in detail. As shown, certain embodiments of the electrosurgical system generally include an electrosurgical probe 20 connected to a power supply 10 for providing high frequency voltage to one or more electrode terminals on probe 20. Probe 20 includes a connector housing 44 at its proximal end, which can be removably connected to a probe receptacle 32 of a probe cable 22. The proximal portion of cable 22 has a connector 34 to couple probe 20 to power supply 10 at receptacle 36. Power supply 10 has an operator controllable voltage level adjustment 38 to change the applied voltage level, which is observable at a voltage level display 40. Power supply 10 also includes one or more foot pedals 24 and a cable 26 which is removably coupled to a receptacle 30 with a cable connector 28. The foot pedal 24 may also include a second pedal (not shown) for remotely adjusting the energy level applied to electrode terminals 42, and a third pedal (also not shown) for switching between an ablation mode and a coagulation mode.

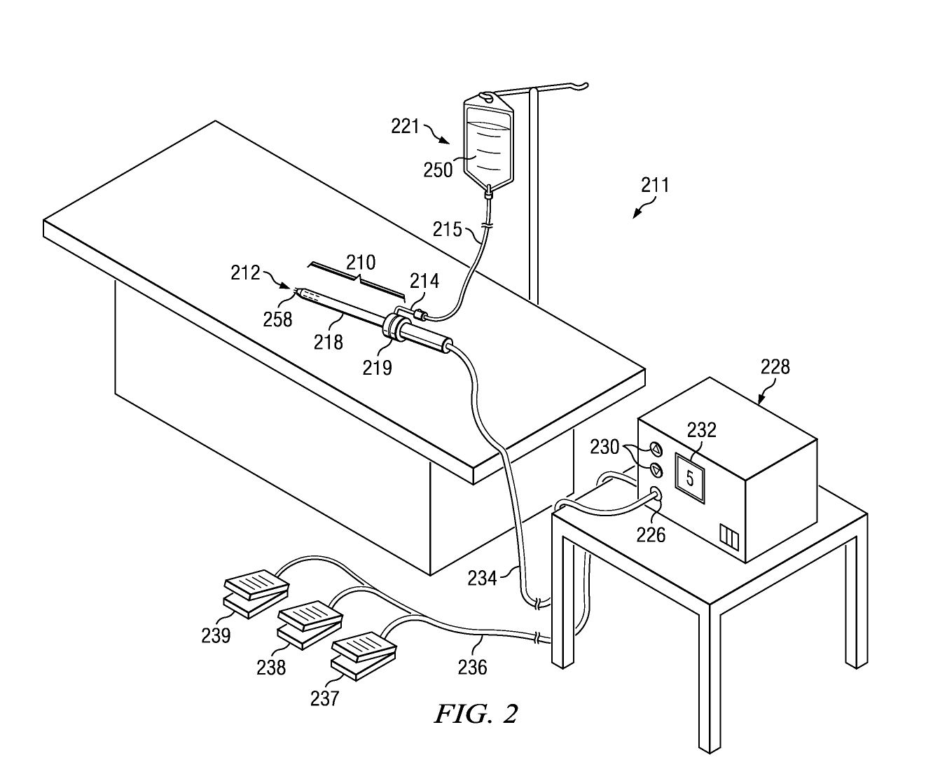

Referring now to FIG. 2, an exemplary electrosurgical system 211 for treatment of tissue in ‘dry fields’ will now be described in detail. Of course, system 211 may also be used in ‘wet field’, i.e., the target site is immersed in electrically conductive fluid. However, this system is particularly useful in ‘dry fields’ where the fluid is preferably delivered through electrosurgical probe to the target site. As shown, electrosurgical system 211 generally comprises an electrosurgical handpiece or probe 210 connected to a power supply 228 for providing high frequency voltage to a target site and a fluid source 221 for supplying electrically conducting fluid 250 to probe 210. The system 211 may also include a vacuum source (not shown) for coupling to a suction lumen disposed in probe 210 (not shown) via a connection tube (not shown) on probe 210 for aspirating the target site, as discussed below in more detail.

As shown, probe 210 generally includes a proximal handle 219 and an elongate shaft 218 having an array 212 of electrode terminals 258 at its distal end. A connecting cable 234 has a connector 226 for electrically coupling the electrode terminals 258 to power supply 228. The electrode terminals 258 are electrically isolated from each other and each of the terminals 258 is connected to an active or passive control network within power supply 228 by means of a plurality of individually insulated conductors (not shown). A fluid supply tube 215 is connected to a fluid tube 214 of probe 210 for supplying electrically conducting fluid 250 to the target site.

Similar to the above embodiment shown in FIG. 1, power supply 228 has an operator controllable voltage level adjustment 230 to change the applied voltage level, which is observable at a voltage level display 232. Power supply 228 also includes first, second and third foot pedals 237, 238, 239 and a cable 236 which is removably coupled to power supply 228. The foot pedals 237, 238, 239 allow the surgeon to remotely adjust the energy level applied to electrode terminals 258. In an exemplary embodiment, first foot pedal 237 is used to place the power supply into the “ablation” mode and second foot pedal 238 places power supply 228 into the “coagulation” mode. The third foot pedal 239 allows the user to adjust the voltage level within the “ablation” mode. In the ablation mode, a sufficient voltage is applied to the electrode terminals to establish the requisite conditions for molecular dissociation of the tissue (i.e., vaporizing a portion of the electrically conductive fluid, ionizing charged particles within the vapor layer and accelerating these charged particles against the tissue). As discussed above, the requisite voltage level for ablation will vary depending on the number, size, shape and spacing of the electrodes, the distance in which the electrodes extend from the support member, etc. Once the surgeon places the power supply in the “ablation” mode, voltage level adjustment 230 or third foot pedal 239 may be used to adjust the voltage level to adjust the degree or aggressiveness of the ablation.

It will be recognized that the voltage and modality of the power supply may be controlled by other input devices. However, applicant has found that foot pedals are convenient methods of controlling the power supply while manipulating the probe during a surgical procedure.

In the coagulation mode, the power supply 228 applies a low enough voltage to the electrode terminals (or the coagulation electrode) to avoid vaporization of the electrically conductive fluid and subsequent molecular dissociation of the tissue. The surgeon may automatically toggle the power supply between the ablation and coagulation modes by alternatively stepping on foot pedals 237, 238, respectively. This allows the surgeon to quickly move between coagulation and ablation in situ, without having to remove his/her concentration from the surgical field or without having to request an assistant to switch the power supply. By way of example, as the surgeon is sculpting soft tissue in the ablation mode, the probe typically will simultaneously seal and/or coagulation small severed vessels within the tissue. However, larger vessels, or vessels with high fluid pressures (e.g., arterial vessels) may not be sealed in the ablation mode. Accordingly, the surgeon can simply step on foot pedal 238, automatically lowering the voltage level below the threshold level for ablation, and apply sufficient pressure onto the severed vessel for a sufficient period of time to seal and/or coagulate the vessel. After this is completed, the surgeon may quickly move back into the ablation mode by stepping on foot pedal 237.

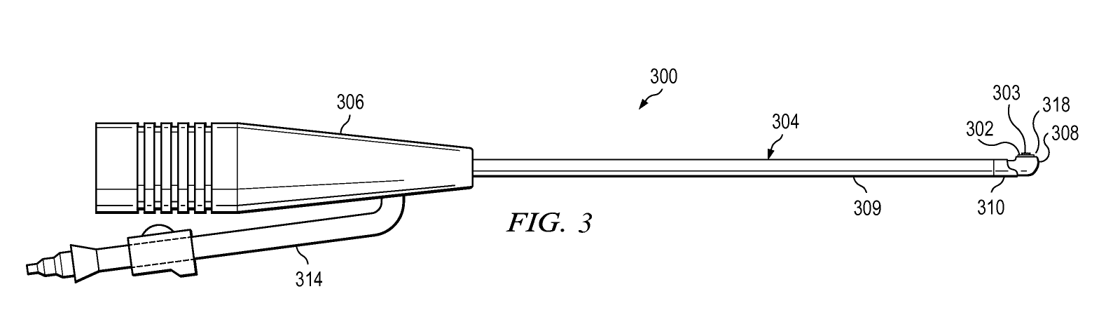

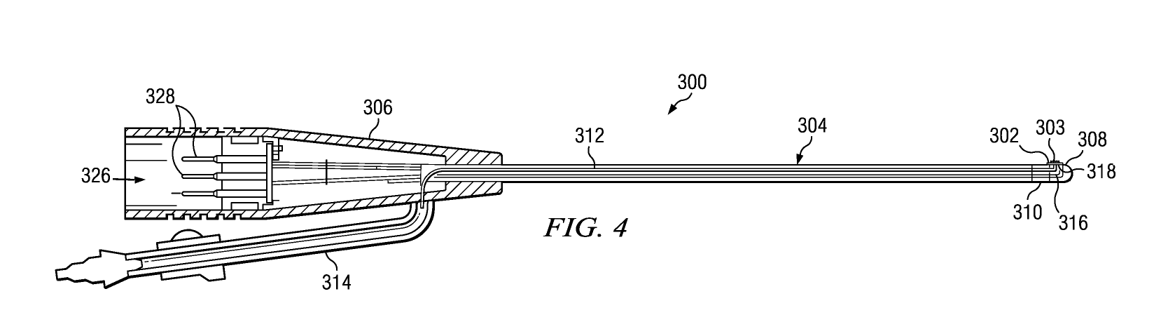

Now referring to FIGS. 3 and 4, an exemplary electrosurgical probe 300 incorporating an active screen electrode 302 is illustrated. Probe 300 may include an elongate shaft 304 which may be flexible or rigid, a handle 306 coupled to the proximal end of shaft 304 and an insulative electrode support member 308 coupled to the distal end of shaft 304. Probe 300 further includes active screen electrode 302 and at least one securing electrode 303. Return electrode 310 is spaced proximally from screen electrode 302 and provides a method for completing the current path back to a power source (similar to those shown in FIGS. 1 and 2). Return electrode 310 may comprise an annular exposed region of shaft 304 slightly proximal of insulative support member 308, typically about 0.5 to 10 mm and more preferably about 1 to 10 mm. Securing electrode 303 and return electrode 310 are each coupled to respective electrical connectors 328 disposed in handle 306 (as illustrated in FIG. 4) that extend to the proximal end of probe 300, where connectors 328 are suitably electrically connected to a power supply (e.g., power supply 10 in FIG. 1 or power supply 228 in FIG. 2). As shown in FIG. 4, handle 306 defines an inner cavity 326 that houses the electrical connectors 328, and provides a suitable interface for connection to an electrical connecting cable (e.g., cable 22 in FIG. 1 or cable 234 in FIG. 2).

Still referencing FIGS. 3 and 4, in certain embodiments screen electrode 302, securing electrode 303 and insulative support member 308 are configured such that screen electrode 302 and securing electrode 303 are positioned on a lateral side of the shaft 304 (e.g., 90 degrees from the shaft axis) to allow the physician to access tissue that is offset from the axis of the portal or arthroscopic opening into the joint cavity in which the shaft 304 passes during the procedure.

Shaft 304 preferably comprises an electrically conducting material, usually metal, which is selected from the group consisting of tungsten, stainless steel alloys, platinum or its alloys, titanium or its alloys, molybdenum or its alloys, and nickel or its alloys. Shaft 304 may include an electrically insulating jacket 309, which is typically formed as one or more electrically insulating sheaths or coatings, such as polytetrafluoroethylene, polyimide, and the like. The provision of the electrically insulating jacket over the shaft prevents direct electrical contact between these metal elements and any adjacent body structure or the surgeon. Such direct electrical contact between a body structure and an exposed electrode could result in unwanted heating and necrosis of the structure at the point of contact causing necrosis.

The probe 300 further includes a suction connection tube 314 for coupling to a source of vacuum, and an inner suction lumen 312 (FIG. 4) for aspirating excess fluids, tissue fragments, and/or products of ablation (e.g., bubbles) from the target site. Preferably, connection tube 314 and suction lumen 312 are fluidly connected, thereby providing the ability to create a suction pressure in lumen 312 that allows the surgeon to draw loose tissue, e.g., synovial tissue, towards the screen electrode 302. Typically, the vacuum source is a standard hospital pump that provides suction pressure to connection tube 314 and lumen 312. As shown in FIGS. 3 and 4, internal suction lumen 312, which preferably comprises peek tubing, extends from connection tube 314 in handle 306, through shaft 304 to an axial opening 316 in support member 308, through support member 308 to a lateral opening 318 in support member 308. Lateral opening 318 may be positioned adjacent to screen electrode 302, which further includes a suction port (not shown) disposed on the surface of screen electrode 302 and fluidly connected to lateral opening 318 for allowing aspiration therethrough.

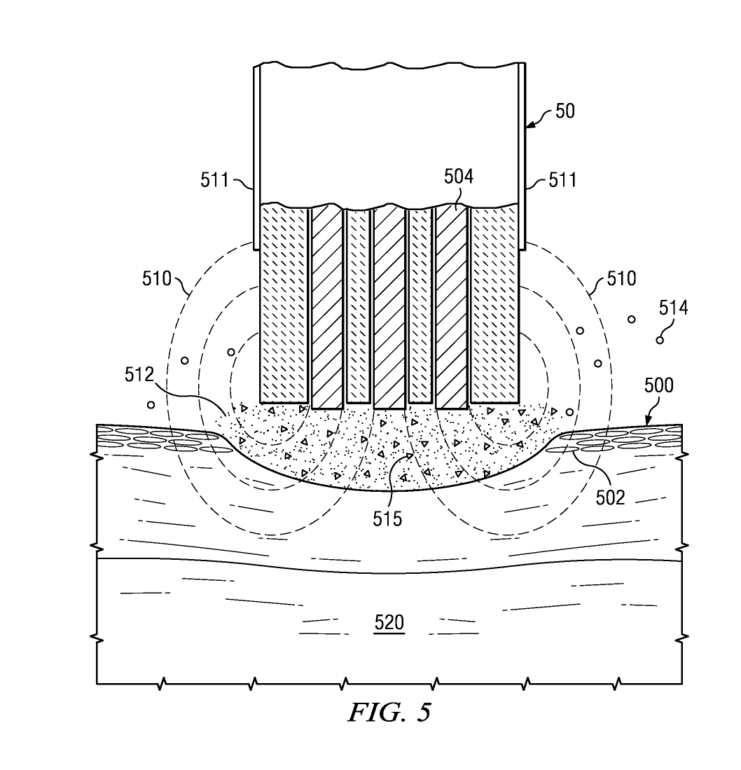

FIG. 5 representatively illustrates in more detail the removal of a target tissue by use of an embodiment of electrosurgical probe 50 according to the present disclosure. As shown, the high frequency voltage is sufficient to convert the electrically conductive fluid (not shown) between the target tissue 502 and active electrode terminal(s) 504 into an ionized vapor layer 512 or plasma. As a result of the applied voltage difference between electrode terminal(s) 504 and the target tissue 502 (i.e., the voltage gradient across the plasma layer 512), charged particles 515 in the plasma are accelerated. At sufficiently high voltage differences, these charged particles 515 gain sufficient energy to cause dissociation of the molecular bonds within tissue structures in contact with the plasma field. This molecular dissociation is accompanied by the volumetric removal (i.e., ablative sublimation) of tissue and the production of low molecular weight gases 514, such as oxygen, nitrogen, carbon dioxide, hydrogen and methane. The short range of the accelerated charged particles 515 within the tissue confines the molecular dissociation process to the surface layer to minimize damage and necrosis to the underlying tissue 520.

During the process, the gases 514 will be aspirated through a suction opening and suction lumen to a vacuum source (not shown). In addition, excess electrically conductive fluid, and other fluids (e.g., blood) will be aspirated from the target site 500 to facilitate the surgeon's view. During ablation of the tissue, the residual heat generated by the current flux lines 510 (typically less than 150° C.) between electrode terminals 504 and return electrode 511 will usually be sufficient to coagulate any severed blood vessels at the site. If not, the surgeon may switch the power supply (not shown) into the coagulation mode by lowering the voltage to a level below the threshold for fluid vaporization, as discussed above. This simultaneous hemostasis results in less bleeding and facilitates the surgeon's ability to perform the procedure. Once the blockage has been removed, aeration and drainage are reestablished to allow the sinuses to heal and return to their normal function.

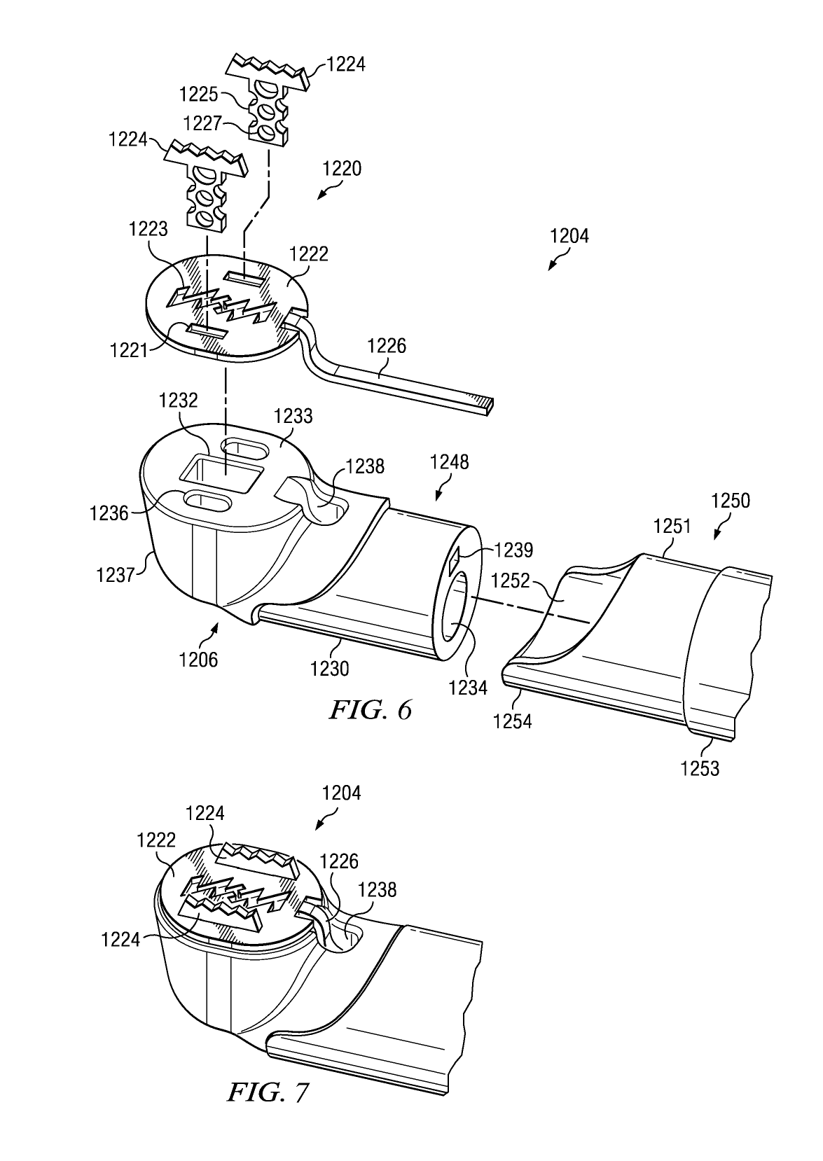

Referring now to FIG. 6, an exploded view of an alternative embodiment of a probe distal end portion 1204 is described, according to at least certain embodiments described in the present disclosure. Probe distal end portion 1204 may include an active electrode assembly 1220, an insulating electrode support member 1230 and a shaft distal end 1250. Shaft distal end 1250 preferably comprises a predominantly metal or electrically conductive material and may provide a portion of an electrical conduit between the shaft distal end 1250 and an energy source (not shown here). In some embodiments, an exposed portion of shaft distal end 1250 may comprise a return electrode 1251, while the remaining portion of the shaft distal end 1250 may have an electrically insulative coating or sheath 1253 to prevent unwanted tissue effect to the patient. Shaft distal end 1250 may have an axial opening 1252, to provide an internal conduit for electrically conductive means such as cables, wires or ribbons. Shaft axial opening 1252 may also provide housing or a protective conduit for fluid lumens, these lumens operable to supply or aspirate fluids, such as electrically conductive fluids, ablative byproducts and patient tissue and fluids to or from the probe distal end portion 1204. Shaft axial opening 1252 may also be formed so as to provide a portion of the structural coupling between the shaft distal end 1250 and insulating electrode support member 1230. As illustrated in FIG. 12, a proximal portion or neck 1248 of support member 1230 may fit within shaft axial opening 1252. Support member 1230 may be press fit into shaft axial opening 1252. Additional or alternative mechanical coupling means may also be used such as adhesives, bolts, clamps or deformable portions such as snaps.

As further illustrated in FIG. 6 shaft axial opening 1252 may have a non uniform or tapered opening, to form a distal extension 1254 of the shaft distal end 1250 on the probe's inferior side 1206. Shaft extension 1254 may improve the mechanical connection between shaft distal end 1250 and support member 1230. Additionally, shaft extension 1254 may provide additional structural support to the probe distal end 1204 during use, such as to add some rigidity to the probe distal end, and provide increased resistance to bending as a user applies force on the active electrode assembly 1220 during use. The inferior shaft extension 1254 may also form a portion of a return electrode 1251, potentially improving the probe's electrosurgical functionality. Generally a return electrode that maintains close proximity to an active electrode assembly allows for reduced power input requirements compared with return electrodes with comparatively larger spacing from an active electrode and consequently, a lower power input may generally decrease the potential for unwanted or widespread tissue effect. Additionally, a return electrode's performance and overall probe performance may be improved when a substantial portion of a return electrode is consistently in fluid contact with a treatment site, so that is it consistently providing a return path to a power supply. Therefore the more distal a portion of the return electrode 1251 is, the more likely the return electrode 1251 will be within a tissue treatment site and the better it may function. Shaft extension 1254 may allow at least a portion or the return electrode 1251 to be disposed closer to or adjacent to the active electrode assembly 1220. A portion of the extension 1254 may preferably axially overlap the electrode assembly 1220.

An electrosurgical probe may also perform better when the spacing between a substantial portion of an active electrode and a substantial portion of a return electrode is consistent, as the tissue effect may be more consistent between the two electrodes. As illustrated in FIG. 12, return electrode 1251 may comprises an annular exposed region of shaft distal end 1250 spaced from active screen electrode 1222, typically about 0.5 mm to 10 mm and more preferably about 1 mm to 3 mm. The curve or taper on shaft extension 1254 as shown may approximately follow an adjacent curved edge on the adjacent electrode assembly 1220 so that there is a more consistent spacing between the electrodes. In this embodiment the proximal side of active screen electrode 1222 curve may approximate the taper on shaft extension 1254.

Insulating electrode support member 1230 preferably comprises an inorganic material, such as glass, ceramic, silicon nitride, alumina, ceramic-filled epoxy, or the like that are substantially resistant to plasma degradation. Support member 1230 may preferably comprise a one piece member with multiple slots, channels or conduits within the member 1230, the one piece member being preferable as it may provide a higher strength component over a multi-piece support member 1230. Support member 1230 may have at least one lateral fluid lumen opening 1232 in communication with an axial fluid lumen opening 1234 to form an internal fluid channel within support member 1230. The at least one lateral fluid opening 1232 may be sized and positioned adjacent to active electrode assembly 1220 to aspirate patient tissue, fluids and gases from the treatment site. Providing suction in the area around treatment may pull patient tissue towards the active electrode assembly 1220 and improve tissue effect, as well as remove much or the debris from the area to improve treatment site visibility. In alternative embodiments, the fluid opening 1232 may supply electrically conductive fluids to the active electrode assembly 1220. Axial fluid opening 1234 may couple with a fluid lumen that extends proximally to deliver patient tissue and aspirated materials to the proximal portion of probe. Axial opening 1234 may be sized to fit standard sized nylon tubing (not shown here).

Support member 1230 may also comprise at least one securing electrode lateral opening or slot 1236 for receiving and potentially coupling with a portion of the at least one securing electrode 1224. A portion of securing electrode 1224 may be secured within said slot 1236 using any number of mechanical fixing means such as adhesives, threaded attachments, snap fits, press fits, interlocking crossbars and polymer press sleeves. As illustrated in FIG. 12 securing electrode 1224 comprises a stem portion 1225 which may have at least one stem aperture 1227. Stem portion 1225 may fit within slot 1236 and be mechanically coupled using adhesive, and the at least one aperture 1227 may improve fixation between the adhesive and slot. Support member 1230 may also comprise a lateral connection element opening 1238 in communication with axial connection element opening 1239, forming a connection element channel or groove in-between that houses an electrically conductive connection element 1226. Support member 1230 may have an axial proximal portion or neck 1248 that is sized to couple with or interface with shaft distal end 1250 using mechanical means as described earlier. Insulating support member distal tip 1237 may be blunt or rounded to provide atraumatic access to treatment site. Support member 1230 may also comprise a flat support surface 1233 for nesting or receiving the active screen electrode 1222. Support surface may have a circumferential ridge or locating pin (not shown) to help retain screen electrode 1222 in place during assembly or during use.

Active electrode assembly 1220 may comprise an active screen electrode 1222, at least one securing electrode 1224 and an electrically conductive connection element 1226 mechanically and electrically connected to the active screen electrode 1222. Wires or electrically conductive means such as ribbons or cables may be electrically coupled to the proximal end of connection element 1226 using electrically coupling means such as soldering, crimping, laser welding or any other means known to those skilled in the art. These wires may extend proximally and internally through the probe shaft to electrical connectors (i.e., electrical connectors 328 in FIG. 4), thereby electrically coupling the screen electrode 1222 to a high frequency power supply. The at least one securing electrode 1224 may be electrically coupled to the active screen electrode 1222 through direct contact with the active screen electrode 1222, or supplemented with other electrically coupling means such as laser welding or soldering. In alternative embodiments, additional internal wiring or electrically conductive means may connect electrical connectors disposed within the probe handle to the at least one securing electrode 1224.

Electrically conductive connection element 1226 preferably comprises an electrically conductive material, such as tungsten, niobium or stainless steel. It may be preferable for the connection element material to be a ductile material so that curves may be more readily formed in the connection element shape and so that the connection element 1226 may easily be inserted into support member lateral opening 1238 and through a channel to axial opening 1239. Connection element 1226 may elastically or non-elastically deform or bend so as to assemble with the support member 1230; however, the connection element 1226 is preferably a sufficiently rigid element so as to provide part of the active screen electrode 1222 securement and limit any screen electrode 1222 motion should other securement points fail. Additionally there is preferably some rigidity to the mechanical and electrical connection between the screen electrode 1222 and connection element 1226, so as to provide some additional movement limitation between the support member 1230 and screen electrode 1222. Insulating support member 1230 may house a portion of the connection element 1226 to secure the electrode assembly 1220 in place and also to electrically insulate the connection element 1226 from the patient tissue and cause any unwanted tissue effect. Additional electrical insulative means may also be used to electrically insulate the connection element 1226, such as shrink tubing or coatings. At least a portion of the electrically conductive connection element 1226 may be formed to preferentially degrade or wear with energy application. A portion of the connection element 1226 may have a reduced width or reduced cross section to create a preferential weak point within the connection element 1226. Alternatively all of the connection element 1226 may be of a substantially consistent width or cross section and the plasma generation may slowly degrade or etch away at a portion of the connection element 1226. This may create a preferable failure mode for the probe, as once a substantial portion the connection element 1226 has been reduced in dimension, energy may no longer be delivered to the active electrode assembly 1220, and the probe will no longer function. However if this failure occurs, the electrode assembly 1220 may still be secured due to other securing points such as the at least one securing electrode 1224.

Active screen electrode 1222 may comprise a conductive material, such as niobium, tungsten, titanium, molybdenum, stainless steel, aluminum, gold, copper or the like. Screen electrode 1222 may have a diameter in the range of about 0.5 mm to 8 mm, preferably about 1 mm to 4 mm, and a thickness of about 0.05 mm to about 2.5 mm, preferably about 0.1 mm to 1 mm. Screen electrode 1222 may have a variety of different shapes, such as the shape shown in FIG. 5. Screen electrode 1222 may have at least one securing electrode aperture or port 1221, and may comprise at least one fluid port 1223 having sizes and configurations that may vary depending on the particular application. Fluid port 1223 may typically be large enough to allow ablated tissue fragments to pass through any downstream suction conduits including lateral fluid opening 1232, axial fluid opening 1234 and fluid lumens extending from these. Fluid port 1223 may preferably be about 2 mm to 30 mm in diameter, preferably about 5 mm to 20 mm in diameter. In some applications, it may be desirable to only aspirate fluid and the gaseous products of ablation (e.g., bubbles) so that the there may be multiple apertures, which may be much smaller, e.g., on the order of less than 10 mm, often less than 5 mm. In certain configurations, fluid port 1223 may be formed in the shape of a zigzag or lightning bolt. Active electrode screen 1222 may be electrically and mechanically coupled to the distal end of connection element 1236 using electrically connecting means such as soldering, crimping, brazing or laser welding. Active electrode screen 1222 may be secured using multiple means as described earlier, and via multiple contact points which is preferable and may improve the general probe robustness and resistance to electrode assembly 1220 dislodgement during use. For example, active screen electrode 1222 may be partially secured via the mechanical connection with the connection element 1226. Additionally, at least one securing electrode 1224 may be used to mechanically secure active screen electrode 1222 in place, as illustrated in FIG. 12. Securing electrode stem 1227 may be mechanically coupled to insulator slot 1236 using an epoxy or ceramic filled adhesive, such as Loctite 3984. Stem apertures 1227 may improve fixation with an adhesive. Stem aperture may also provide some space within lateral opening 1236 for any expansion or thermal shock reactions the stem or adhesive may have to the energy being applied. In alternative embodiments, securing electrode stems may have teeth or roughened surfaces, or snaps or cross bars (not shown) to improve mechanical fixation. The at least one securing electrode 1224 may further be coupled to the screen electrode 1222 using mechanical means such as laser welding, brazing, soldering or using adhesives, preferably electrically conductive adhesives.

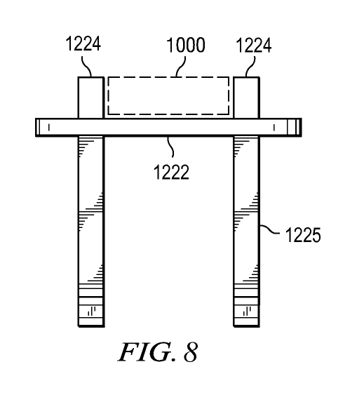

FIG. 7 illustrates an assembled representation of distal end portion 1204 of embodiment as described in FIG. 3. Distal end portion 1204 of a representative probe is shown with at least two bilateral securing electrodes 1224 thereon, said securing electrode 1224 providing both a portion of the tissue treatment and securement of screen electrode 1222. In this configuration, securing electrodes 1224 may be oriented symmetrically about the central axis of shaft distal end 1250, and may thereby allow for creation of a zone for RF ablation or plasma chamber between the symmetrically oriented bilateral securing electrodes 1224 as well as between securing electrodes 1224 and screen electrode 1222 (as shown in FIG. 8). Incorporation of symmetrical securing electrodes 1224 may allow for the creation of a three dimensional zone represented by plasma zone 1000 shown in FIG. 8 for carrying out RF ablation. The placement of the bilateral securing electrodes 1224 not only provides for mechanical fixation but also provides a plasma forming feature. The securing electrodes surface may have a saw tooth, serrated, or rasp-like feature for enhanced plasma formation and increased tactile feedback during soft tissue ablation. FIG. 7 also shows the assembly of connection element 1226 within connection element lateral opening 1239 for additional screen electrode securement.

Other modifications and variations can be made to disclose embodiments without departing from the subject invention as defined in the following claims. For example, it should be noted that the invention is not limited to an electrode array comprising a plurality of electrode terminals. The invention could utilize a plurality of return electrodes, e.g., in a bipolar array or the like. In addition, depending on other conditions, such as the peak-to-peak voltage, electrode diameter, etc., a single electrode terminal may be sufficient to contract collagen tissue, ablate tissue, or the like.

In addition, the active and return electrodes may both be located on a distal tissue treatment surface adjacent to each other. The active and return electrodes may be located in active/return electrode pairs, or one or more return electrodes may be located on the distal tip together with a plurality of electrically isolated electrode terminals. The proximal return electrode may or may not be employed in these embodiments. For example, if it is desired to maintain the current flux lines around the distal tip of the probe, the proximal return electrode will not be desired.

While preferred embodiments of this invention have been shown and described, modifications thereof can be made by one skilled in the art without departing from the scope or teaching herein. The embodiments described herein are exemplary only and are not limiting. Because many varying and different embodiments may be made within the scope of the present teachings, including equivalent structures or materials hereafter thought of, and because many modifications may be made in the embodiments herein detailed in accordance with the descriptive requirements of the law, it is to be understood that the details herein are to be interpreted as illustrative and not in a limiting sense.

What is claimed is:

1. An electrosurgical instrument for applying energy to target site within or on a patient's body, comprising:
a shaft having a proximal end and a distal end;
an electrically insulating support member disposed adjacent the shaft distal end;
an active electrode assembly comprising a substantially flat active screen electrode disposed on said support member and at least one securing electrode in electrical communication with the screen electrode and at least one electrically conductive connection element electrically coupled to the screen electrode, wherein the connection element provides a portion of an electrical conduit between the active electrode assembly and an energy source; and
wherein the support member has at least one securing electrode slot to securedly engage with a portion of the at least one securing electrode and wherein the support member also has at least one conductive connection element opening to receive a portion of the connection element.

2. The instrument of claim 1, further comprising at least one return electrode positioned on the shaft and spaced away from the active electrode assembly.

3. The instrument of claim 2, wherein the return electrode further comprises an inferior extension, wherein a portion of the inferior extension axially overlaps a portion of the electrode assembly.

4. The instrument of claim 1, comprising at least two securing electrodes.

5. The instrument of claim 1, wherein the screen electrode comprises at least one securing electrode opening to receive a portion of the securing electrode.

6. The instrument of claim 1 wherein the electrically conductive connection element further comprises a preferential wear portion, operable to be preferentially etched during the application of plasma energy.

7. The instrument of claim 1 wherein the electrically conductive connection element is formed from an electrically conductive material, selected from a group consisting of Stainless Steel, Tungsten, Titanium, Niobium, and Molybdenum.

8. The instrument of claim 1, wherein the connection element comprises a ductile material, so that the connection element may flex during assembly.

9. The instrument of claim 1, wherein the at least one securing electrode has a stem portion, and wherein the stem portion is mechanically fixed with the at least one support member securing electrode slot using an adhesive.

10. The instrument of claim 9, wherein the at least one stem portion further comprises at least one lateral aperture, operable to improve flow of the adhesive and thereby improve mechanical fixation between the stem portion and the support member.

11. The instrument of claim 1, wherein the at least one securing electrode has a saw tooth pattern on a superior surface.

12. The instrument of claim 1 further comprising an aspiration element, comprising at least one lumen disposed within the shaft, in fluid communication with at least one support member aspiration channel disposed within the insulating support member, and at least one aspiration aperture on the screen electrode in fluid communication with said support member aspiration channel.

13. The instrument of claim 1, wherein the insulative support member comprising a one piece component, comprising a material operable to be resistant to electrosurgical plasma degradation.

14. The instrument of claim 2, wherein upon the application of a sufficiently high frequency voltage between the screen electrode, the at least one securing electrode, and the return electrode to vaporize the conductive fluid in a thin layer over at least a portion of the screen electrode and the at least one securing electrode and to induce the discharge of energy from the vapor layer.

15. A method for securing a substantially flat active screen electrode to a distal end of an electrosurgical instrument shaft comprising:
electrically connecting an electrically conductive connection element to the screen electrode;
inserting the connection element into an electrically insulating support member connection element channel;
disposing the screen electrode upon the electrically insulating support member;
extending a stem portion of a securing electrode through a port in the screen electrode and into a slot in the electrically insulating support member;
fixedly engaging the stem portion of the securing electrode within the slot in the electrically insulating support member, the securing electrode mechanically securing the screen electrode to the support member and in electrical contact with the screen electrode;
electrically coupling the connection element to an electrical connector, wherein the electrical connector is connected with a high frequency power supply; and
mechanically connecting the electrically insulating support member to a distal end of the shaft of an electrosurgical instrument.

16. The method of claim 15, wherein the screen electrode and the securing electrode are prevented from moving axially and laterally with respect to the shaft of the instrument.

17. The method of claim 15, wherein the step of fixedly engaging comprises securing the stem to the support member with an epoxy.

18. The method of claim 15 wherein the step of inserting comprises bending the connection element.

19. An electrosurgical instrument for applying energy to a target site within or on a patient's body, comprising:
a shaft having a proximal end and a distal end;
an insulating support member disposed adjacent to the shaft distal end, wherein the support member comprises a screen electrode support surface, at least one securing electrode opening disposed on the screen electrode support surface, and at least one connection element channel adjacent the screen electrode support surface;
an active electrode assembly comprising a substantially flat active screen electrode disposed on the screen electrode support surface and at least one securing electrode, wherein a portion of the at least one securing electrode is disposed within the securing electrode opening; and
at least one electrically conductive connection element in electrical communication with the screen electrode, wherein a portion of the connection element is disposed within the connection element channel.

20. The electrosurgical instrument of claim 19 wherein the insulating support member further comprising a proximal neck operable to fit within and be secured to shaft distal end.

21. The electrosurgical instrument of claim 19, wherein the insulating support member further comprises a support member fluid channel in fluid communication with a fluid lumen disposed within the instrument shaft and also in fluid communication with a screen electrode fluid port.

22. The instrument of claim 19, wherein the insulative support member comprises a one piece component,

23. The instrument of claim 22, wherein the insulative support member comprises an inorganic material resistant to electrosurgical plasma degradation.
